(19) United States
(12) Patent Application Publication
(10) Pub. No.: US 2008/0140001 A1
(43) Pub. Date: Jun. 12, 2008

(54) Fluid Delivery Apparatus And Methods

(75) Inventors: Oren Globerman, Kfar-Shemaryahu (IL); Mordechay Beyar, Caesarea (IL); Rami Keller, Tel-Aviv (IL); Eran Schwimmer, Herzlia (IL); Inbal Shraga, Herzlia (IL)

Correspondence Address: Martin MOYNIHAN;PRTSI, Inc., P.O. Box 16446, Arlington, VA, 22215, US

(73) Assignee: By-Pass Inc., Orangeburg, NY (US)

(21) Appl. No.: 11/609,451

(22) Filed: Dec. 12, 2006

(51) Int. Cl. A61M 29/00

(52) U.S. Cl. 604/97.01

(57) ABSTRACT

A pressure sensitive valve comprising: (a) an outer balloon adapted for intravascular insertion comprising at least one hole adapted for ejection of a fluid; (b) an inner structure adapted to substantially fill the outer balloon; (c) at least one selectively blockable flow path between the outer balloon and the inner structure, at least some of the at least one flow path in fluid communication with at least one of the at least one hole; (d) an inlet port to the at least one flow path; and (e) a pressure source operable to provide a fluid at least at a selected injection pressure to the inlet port. A flow of the fluid along the at least one selectively blockable flow path to the at least one hole is prevented when the pressure source provides any pressure below the selected injection pressure and a flow of the fluid along the at least one selectively blockable low path to the at least one hole occurs when the pressure source provides pressure at or above the selected injection pressure.

### RELATED APPLICATIONS

This application is related to U.S. Application 2006/0190022 entitled “Transvascular Ablation System” and filed on Jan. 19, 2006 and to PCT application WO 2006/006169 filed Jul. 14, 2005 and entitled “Material Delivery System”. The disclosures of these applications are each fully incorporated herein by reference.

### FIELD OF THE INVENTION

The present invention relates to apparatus and methods for fluid delivery.

### BACKGROUND OF THE INVENTION

A common treatment for stenosis is PTCA in which a balloon is inflated to compress the blockage and/or forcefully expand the artery. Restenosis and arterial collapse are common problems with this approach.

It has been previously suggested to inject various types of medication into the site of a stenosis at high velocity. However, there are not currently any known commercially available devices which take advantage of the high injection velocity strategy.

U.S. Pat. No. 5,611,775, the disclosure of which is incorporated herein by reference, describes prevention of restenosis and/or arterial collapse by injecting drugs into the blood vessel wall using a balloon with small holes deployed at the site of stenosis. Specifically, this patent describes inflating an inner balloon to force the drug through holes in an outer balloon.

U.S. Pat. No. 5,614,502 entitled “High pressure impulse transient drug delivery for the treatment of proliferative diseases” and U.S. Pat. No. 6,716,190 entitled “Device and method for the delivery and injection of therapeutic and diagnostic agents to a target site within a body”, the contents of which are incorporated herein by reference, describe methods of material delivery inside the body, including transvascularly.

W. J. Walker, I. M. Faireley “A simplified technique for the per catheter delivery of Isobutyl 2—Cyanoacrylate in the Embolisation of Bleeding Vessels”, Journal of Interventional Radiology 1987 2, 59-63, the contents of which is incorporated herein by reference, describes the injection of glue into a lumen and against walls of an artery, in order to block it.

U.S. Pat. No. 6,280,414, the disclosure of which is incorporated herein by reference describes a tube system for delivering a drug to the wall of a blood vessel.

U.S. Pat. Nos. 5,730,723 and 5,704,911, the disclosures of which are fully incorporated herein by reference, describe needleless injection apparatus.

### SUMMARY OF THE INVENTION

A broad aspect of some embodiments of the invention relates to delivery of a fluid at a high velocity into tissue surrounding an intrabody lumen. In an exemplary embodiment of the invention, the intrabody lumen is a blood vessel, optionally a coronary blood vessel.

An aspect of some embodiments of the invention relates to a pressure sensitive valve which includes a valve lumen that expands to accommodate a fluid delivered at a sufficient pressure. The valve opens at, or above, the sufficient pressure to allow the delivered fluid to escape through one or more holes in a wall of the valve lumen. The sufficient pressure is referred to herein as “P injection” (Pᵢₙⱼ). Pᵢₙⱼ is a minimum value and injection continues as long as pressure in the valve lumen remains at or above Pᵢₙⱼ. In an exemplary embodiment of the invention, Pᵢₙⱼ is an adjustable parameter. Optionally, adjustment can be integral in valve design or user configurable during valve use. In an exemplary embodiment of the invention, one or more of valve volume and an elasticity coefficient “K” of the wall of the valve lumen contribute to Pᵢₙⱼ. Optionally, K can be constant or variable. In an exemplary embodiment of the invention, the valve lumen is elastic only at a certain range of volumes or shape/volume combinations.

In an exemplary embodiment of the invention, a volume of the lumen at pressures below Pᵢₙⱼ is substantially zero. Optionally, a portion of the valve lumen is occupied by an inner structure. In an exemplary embodiment of the invention, the inner structure is a balloon. Optionally, the inner balloon is inflated to occupy substantially all of the lumen. In an exemplary embodiment of the invention, the inner balloon is inflated to a loading pressure which would cause it to burst if it were not supported externally by the wall of the valve lumen.

In some exemplary embodiments of the invention, the valve lumen comprises an outer balloon which substantially conforms to the inner structure. Optionally, the inner structure is a balloon which causes the conformation when inflated or an inelastic structure with a fixed volume. In an exemplary embodiment of the invention, conformation of the outer balloon to the inner structure covers the hole(s) directly. In an exemplary embodiment of the invention, valves of this general configuration are re-usable.

In an exemplary embodiment of the invention, the valve comprises an outer balloon which defines the valve lumen and is perforated by one or more holes and also comprises an inner structure occupying a portion of the valve lumen defined by the outer balloon. Optionally, the inner structure is an inner balloon which is inflatable independently of the outer balloon or a rigid structure with a fixed volume. According to these exemplary embodiments, the inner structure blocks a flow of fluid through the lumen of the outer balloon to the hole(s) at pressures below Pᵢₙⱼ. In an exemplary embodiment of the invention, there is a potential advantage to inflating an inner structure comprising a balloon to insure contact of the outer balloon with an external tissue (e.g. a blood vessel).

In exemplary embodiments of the invention which comprise an outer balloon and an inner balloon, each of the balloons is independently characterized by a coefficient of elasticity “K”. In an exemplary embodiment of the invention, as the inner balloon is inflated, pressure in the inner balloon overcomes the K of the inner balloon and the inner balloon expands. As the inner balloon expands it overcomes the K of the outer balloon. At this stage, both balloons are inflated and substantially all of the volume of a lumen of the outer balloon is occupied by the inner balloon. In this way, the inner balloon serves to “pre-load” the outer balloon with a pressure while keeping holes in the outer balloon sealed. In an exemplary embodiment of the invention, K of the outer balloon is 500, 600, 700, 800, 900 or 1000 N/mm of additional diameter or lesser or intermediate or greater values. In an exemplary embodiment of the invention, K of the outer balloon is greater than K of the inner balloon by a factor of 2, 3, 5, or 10 or lesser or intermediate or greater values.

In an exemplary embodiment of the invention, Pᵢₙⱼ is equal to a pre-loading pressure of the inner balloon minus (K of the inner balloon times a constant).

In some exemplary embodiment of the invention, the lumen of the outer structure is provided as one or more channels. Optionally, the channels can be produced in a variety of ways. One exemplary way to produce channels is to provide the inner and/or outer structure with ribs which contact a surface of an opposing structure and form channels which are in fluid communication with the holes of the outer structure.

In an exemplary embodiment of the invention, a flow of fluid through the channels is blocked below Pᵢₙⱼ by a separate element, as opposed to contact between the inner and outer structures. Optionally, the separate element can be configured as rupture discs, snap-valves or spring actuated valves. In an exemplary embodiment of the invention, a valve containing separate elements configured as rupture discs or snap valves are single use valves. In an exemplary embodiment of the invention, a valve containing separate elements configured as spring actuated valves can be re-usable. In an exemplary embodiment of the invention, the valve is configured as a normally closed valve in which the hole(s) are closed when pressure in the valve lumen is below Pᵢₙⱼ.

An aspect of some embodiments of the invention relates to a method of delivering material to target cells surrounding an intrabody lumen. In an exemplary embodiment of the invention, the method includes positioning a pressure sensitive valve in proximity to the target cells, creating a resistance pressure in a valve lumen and supplying fluid to the valve lumen from a pressure source outside the body at sufficient pressure to overcome the resistance pressure. When the resistance pressure is overcome, fluid exits the valve at high velocity and is injected into tissue.

An aspect of some embodiments of the invention relates to re-shaping a pressure pulse conveyed through a conduit from a proximal end to a distal end by providing a pressure sensitive valve at the distal end of the conduit. In an exemplary embodiment of the invention, the valve is configured as a balloon within a balloon. Optionally, a pressure pulse provided at a proximal end of the conduit spreads out as it travels through the conduit and is re-sharpened at a distal end of the conduit by the pressure sensitive valve. In an exemplary embodiment of the invention, the valve responds by opening in 5, 10, 20 or 50 milliseconds or lesser or greater or intermediate times when the pressure pulse arrive to the distal end of the conduit. Optionally, there is a trade-off between a time duration of the pressure pulse and a degree of re-shaping.

An aspect of some embodiments of the invention relates to providing an acceleration path for molecules of liquid propelled by pressure of a sufficient magnitude.

In an exemplary embodiment of the invention, the path includes a space between an outer balloon with one or more holes and an inner balloon which becomes available at Pᵢₙⱼ or greater.

In an exemplary embodiment of the invention, the path includes holes in an outer balloon which open at or above an injection pressure. Optionally, the holes are characterized by a different size and/or shape at an outer surface than at an inner surface of the balloon. Optionally, the path comprises substantially only the holes in the outer balloon.

In an exemplary embodiment of the invention, the path becomes available because the outer balloon expands and/or the inner balloon contracts.

In an exemplary embodiment of the invention, the path includes one or more elongate channels fitted with microvalves which open at Pᵢₙⱼ or greater. In an exemplary embodiment of the invention, the acceleration path can be as short as, for example, 5, 10, 20, 50 or 100 microns and/or as long as 5, 10, 15, 20 or 25 mm long or shorter or intermediate or greater lengths.

In an exemplary embodiment of the invention, fluid exits the holes with a velocity of 3, 5, 8 or 10 M/s or lesser or greater or intermediate velocities. Optionally, some fluid leaks from the valve at pressures below Pᵢₙⱼ. In an exemplary embodiment of the invention, the leaking occurs at low velocity.

In an exemplary embodiment of the invention, there is provided a pressure sensitive valve, the valve comprising:

(a) an outer balloon adapted for intravascular insertion comprising at least one hole adapted for ejection of a fluid;

(b) an inner structure adapted to substantially fill the outer balloon;

(c) at least one selectively blockable flow path between the outer balloon and the inner structure, at least some of the at least one flow path in fluid communication with at least one of the at least one hole;

(d) an inlet port to the at least one flow path; and

(e) a pressure source operable to provide a fluid at least at a selected injection pressure to the inlet port;

wherein a flow of the fluid along the at least one selectively blockable flow path to the at least one hole is prevented when the pressure source provides any pressure below the selected injection pressure; and

wherein a flow of the fluid along the at least one selectively blockable low path to the at least one hole occurs when the pressure source provides pressure at or above the selected injection pressure.

Optionally, the outer balloon is elastic.

Optionally, a coefficient of elasticity “K” of the outer balloon is at least 500 N/mm.

Optionally, the inner structure comprises a balloon.

Optionally, the balloon is elastic.

Optionally, a coefficient of elasticity “K” of the outer balloon is at least 100% greater than a coefficient of elasticity of the inner balloon.

Optionally, the outer balloon conforms to the inner structure at any pressure below the selected injection pressure.

Optionally, the outer balloon expands when pressure at the inlet port reaches or exceeds the selected injection pressure.

Optionally, the valve is provided as a portion of an atherectomy catheter.

In an exemplary embodiment of the invention, there is provided a method of delivering fluid to tissue surrounding an intrabody lumen at a high velocity, the method comprising:

(a) inserting a pressure sensitive valve into an intrabody lumen, the valve configured to prevent a flow of fluid from one or more holes at any pressure below a selected injection pressure and to permit the flow at the selected injection pressure or greater; and

(b) delivering a fluid pulse to the valve at the selected injection pressure or greater.

Optionally, the valve is adjacent to one or more holes.

Optionally, the valve comprises one or more holes.

Optionally, the fluid pulse comprises a liquid medication.

Optionally, exit of a volume not exceeding 0.25 ml reduces pressure in the valve below the selected injection pressure.

Optionally, the selected injection pressure is at least 10 atmospheres.

Optionally, delivering fluid to the valve at the selected injection pressure or greater is repeated and the flow is prevented between repetitions.

Optionally, the method comprises adjusting an ejection direction between repetitions.

Optionally, the method comprises adjusting a position of the valve between the repetitions.

Optionally, the method is performed in conjunction with a stenosis therapy procedure.

Optionally, the stenosis therapy procedure comprises atherectomy.

Optionally, the stenosis therapy procedure comprises PTCA.

In an exemplary embodiment of the invention, there is provided a method of delivering fluid to tissue surrounding an intrabody lumen at a high velocity, the method comprising:

(a) inserting a pressure sensitive valve comprising an outer balloon with one or more holes and an inner balloon into an intrabody lumen;

(b) inflating the inner balloon so that it conforms to the outer balloon; and

(c) causing fluid to flow into a lumen of the outer balloon at a sufficient pressure to cause at least a portion of the fluid to exit the balloon through the holes at a velocity sufficient to penetrate surrounding tissue while the inner balloon remains inflated.

Optionally, (a) occurs first, (b) occurs second and (c) occurs third.

Optionally, the method comprises inflating the inner balloon to open a stenosis. Optionally, the method comprises reducing pressure in the inner balloon after opening the stenosis.

In an exemplary embodiment of the invention, there is provided a method of delivering fluid to tissue surrounding an intrabody lumen at a high velocity, the method comprising:

(a) stopping molecules of liquid propelled by an increasing pressure approaching a selected injected pressure, the increasing pressure supplied from a pressure source outside the body, using a pressure sensitive valve installed in a body lumen; and

(b) opening an acceleration path for the molecules when the increasing pressure reaches or exceeds the selected injection pressure

Optionally, the valve stops the molecules within the valve.

Optionally, the valve stops the molecules prior to entry into the valve.

Optionally, opening the acceleration path comprises stretching an elastic membrane.

Optionally, the stretching an elastic membrane comprises expanding an elastic balloon.

Optionally, opening the acceleration path comprises deforming a plastically deformable element.

Optionally, opening the acceleration path comprises deforming an elastically deformable element.

Optionally, opening the acceleration path comprises opening one or more elongate channels by operating one or more microvalves which open at the selected injection pressure.

In an exemplary embodiment of the invention, there is provided a pressure sensitive valve, the valve comprising:

(a) a biocompatible unit, the unit adapted for insertion in an intrabody lumen;

(b) at least one acceleration path for a fluid, each of the at least one acceleration path terminating in at least one hole facing outwards from the biocompatible unit;

(c) an inlet port to the at least one flow path;

(d) a pressure source operable to provide fluid at a selected injection pressure to the inlet port; and

(e) at least one flow restriction element adapted to:
(i) block a flow of the fluid along the at least one flow path at any pressure below the selected injection pressure; and
(ii) permit a flow of the fluid along the at least one flow path at the selected injection pressure or greater.

In an exemplary embodiment of the invention, there is provided a liquid drug delivery device for treating intra-body lumen tissues, the device comprising:

an inflatable inner structure containing a fluid at a substantially constant inner threshold pressure;

an outer structure having at least one hole, adapted for ejecting the drug;

a volume between said structures containing a liquid drug at a first pressure, which is substantially equivalent to said threshold pressure; and

a pressure pulse source having direct communication with said volume, adapted to substantially increase the pressure in said volume, when activated, for a period not exceeding 100 milliseconds;

wherein said inner structure seals said at least one hole when said pressure pulse source is not activated.

### BRIEF DESCRIPTION OF THE DRAWINGS

Exemplary non-limiting embodiments of the invention described in the following description, read with reference to the figures attached hereto. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIGS. 1A and 1B are flow diagrams illustrating exemplary methods according to some embodiments of the invention;

FIG. 2A is a schematic diagram of a fluid delivery system according to an exemplary embodiment of the invention;

FIGS. 2B and 2D are cross sections of exemplary catheters according to embodiments of the invention at line B-B of FIG. 2A;

FIG. 2C is a cross section of an exemplary catheter according to an embodiment of the invention at line A-A of FIG. 2A;

FIGS. 3A, 3B, 3C and 3D are schematic diagrams illustrating an operational sequence of a pressure sensitive valve according to an exemplary embodiment of the invention;

FIGS. 3E and 3F are lateral and transverse cross sections respectively of a pressure sensitive valve according to another exemplary embodiment of the invention;

FIGS. 4A and 4B are schematic diagrams of a “fluid gun” according to an exemplary embodiment of the invention in “cocked” and “fired” states respectively;

FIGS. 5A, 5B, 5C and 5D illustrate exemplary arrangements of holes on a balloon according to exemplary embodiments of the invention;

FIG. 6 is a micrograph of tissue illustrating penetration of material injected using an apparatus according to an exemplary embodiment of the invention;

FIGS. 7 and 8 are cross sectional views of additional exemplary embodiments of pressure sensitive valves according to the invention;

FIG. 9 is a graph illustrating internal pressure profiles of an inner balloon (dotted line) and outer balloon (solid line) during an exemplary injection event according to one embodiment of the invention; and

FIGS. 10A and 10B are diagrams illustrating operation of opposing forces in valves according to exemplary embodiments of the invention.

### DESCRIPTION OF EXEMPLARY EMBODIMENTS

Overview

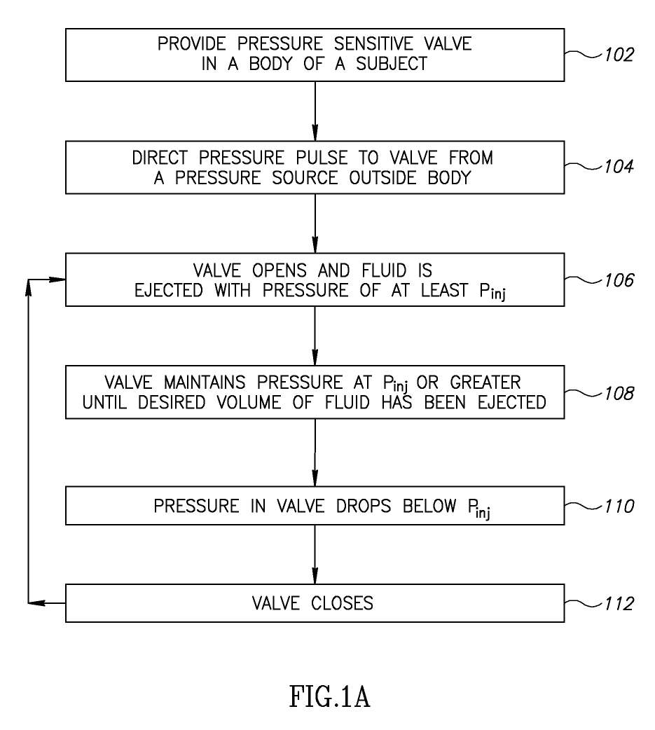

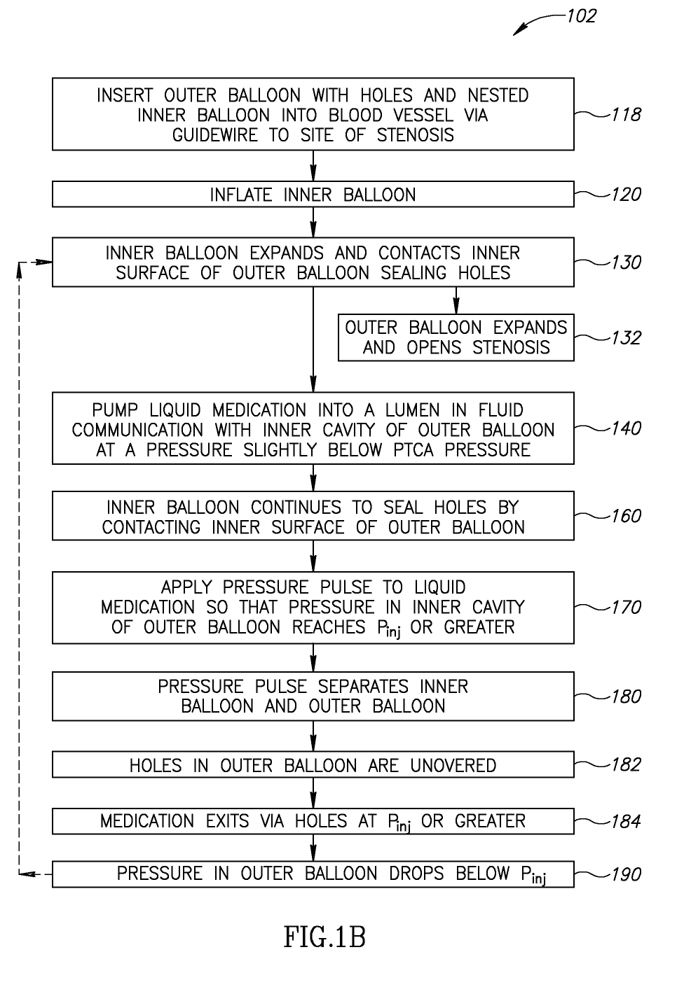

FIG. 1A is a simplified flow diagram illustrating an exemplary method of injecting a therapeutic agent into tissue surrounding an intrabody lumen.

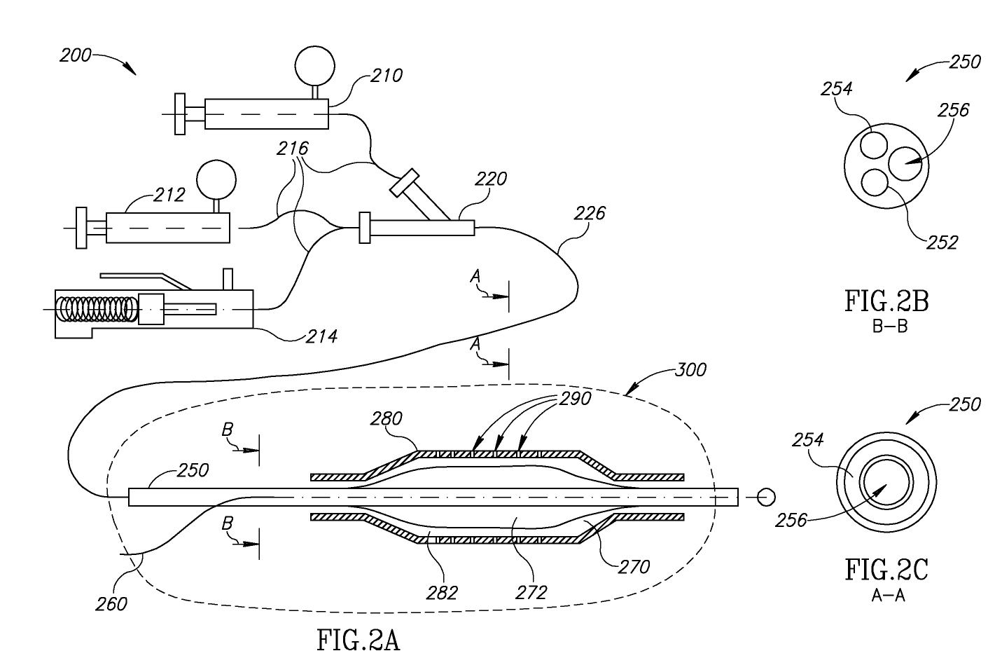

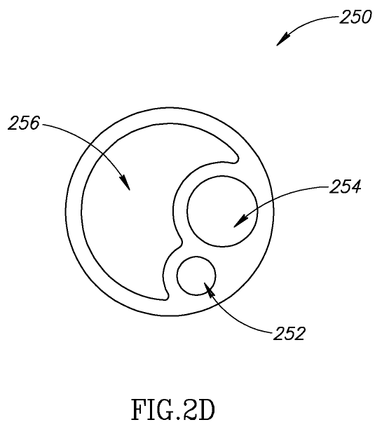

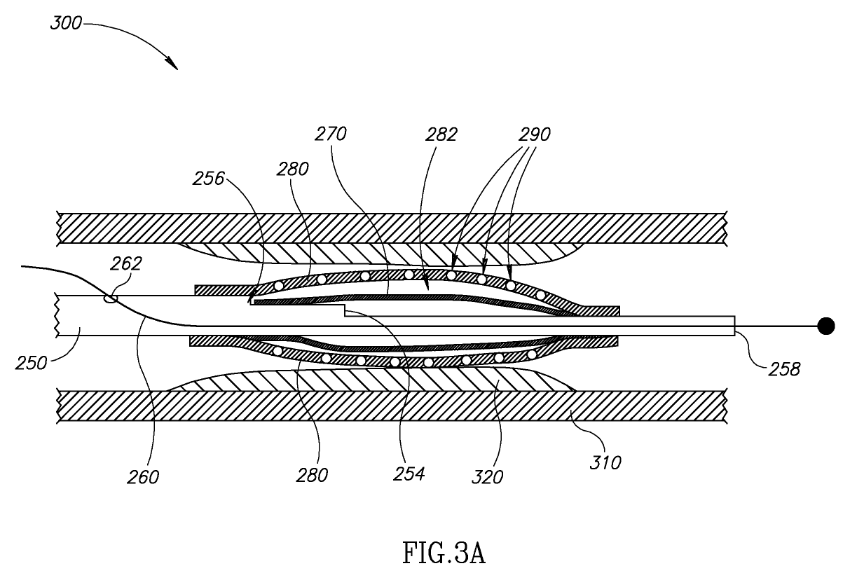

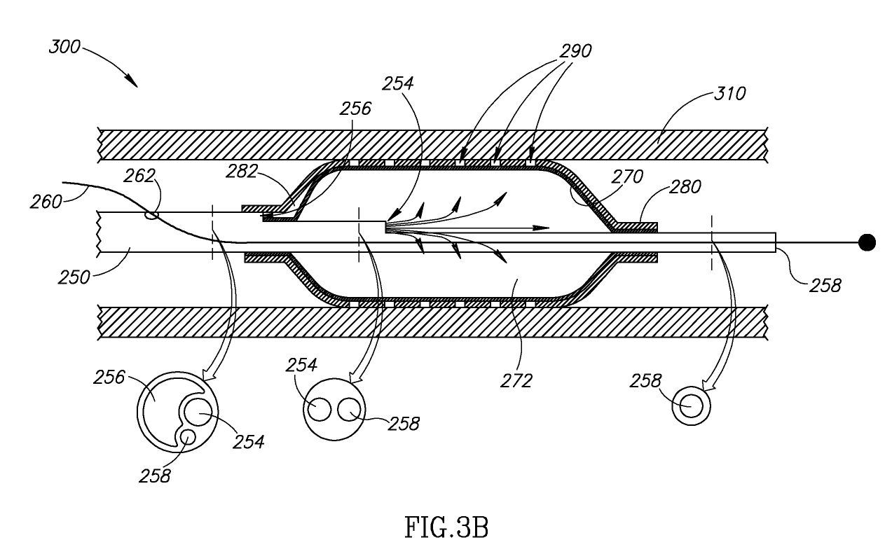

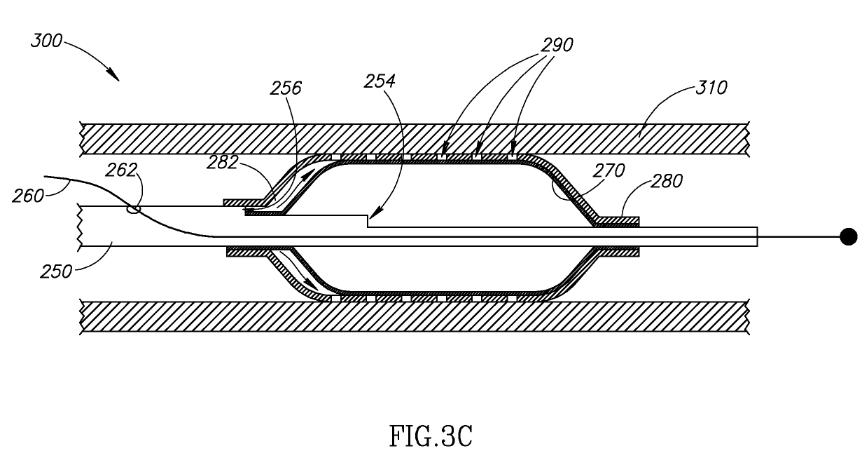

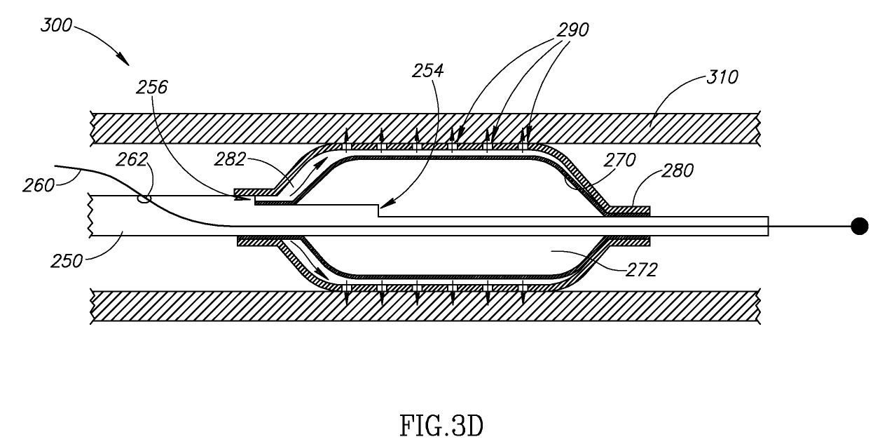

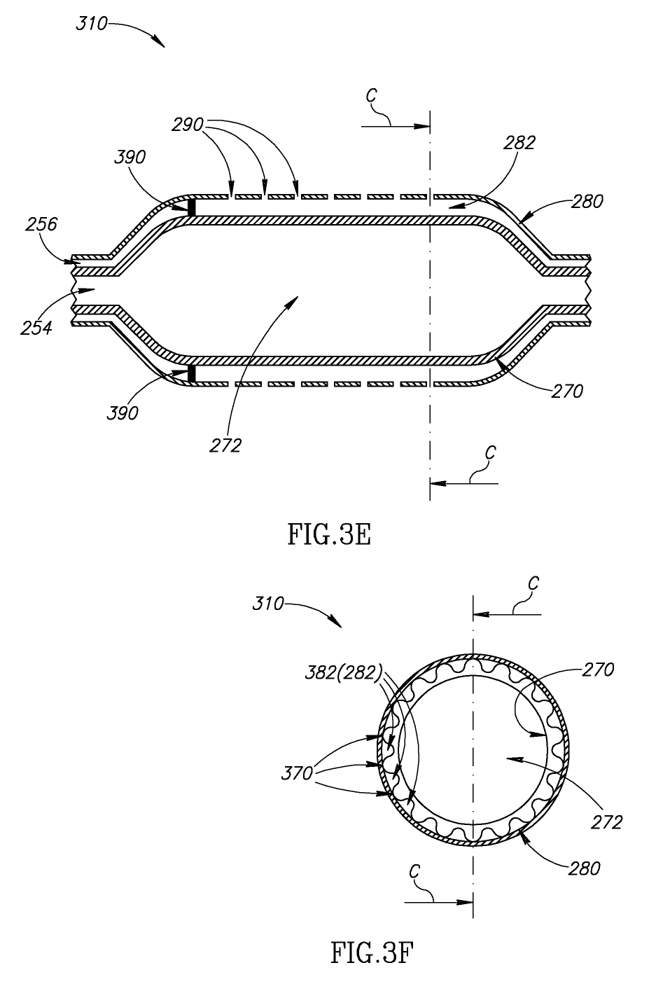

FIG. 2A is a schematic diagram of an exemplary system 200 configured to perform exemplary method 100. System 200 is depicted as including a pressure sensitive valve 300 which is depicted in greater detail in FIGS. 3A, 3B, 3C and 3D which are described below. Exemplary valve 300 is a double chambered device characterized by an inner chamber and an outer chamber. Other components of system 200 serve to independently regulate internal pressures of the inner and outer chambers.

In other exemplary embodiments of system 200, valve 300 is replaced by a valve with a different configuration. Exemplary alternate valve configurations 302 and 304 are depicted in FIGS. 3E-3F, 7 and 8 respectively and will be described below. According to various exemplary embodiments of the invention, the pressure sensitive valve is configured according to the specific application for which it is designed.

Referring now to FIGS. 1A and 2A operation of exemplary valve 300 is explained:

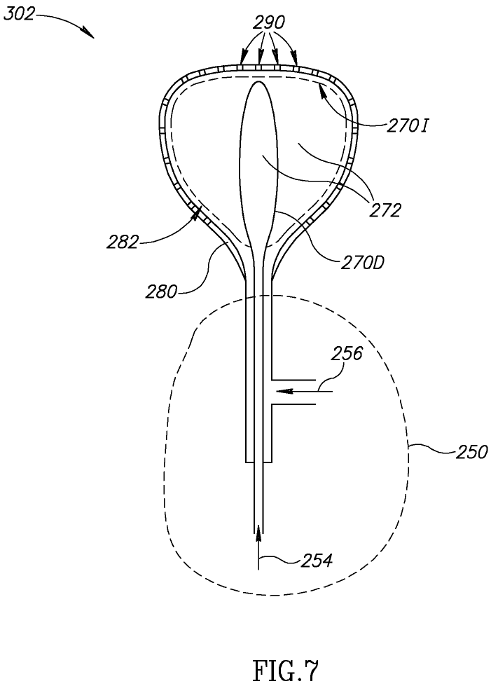

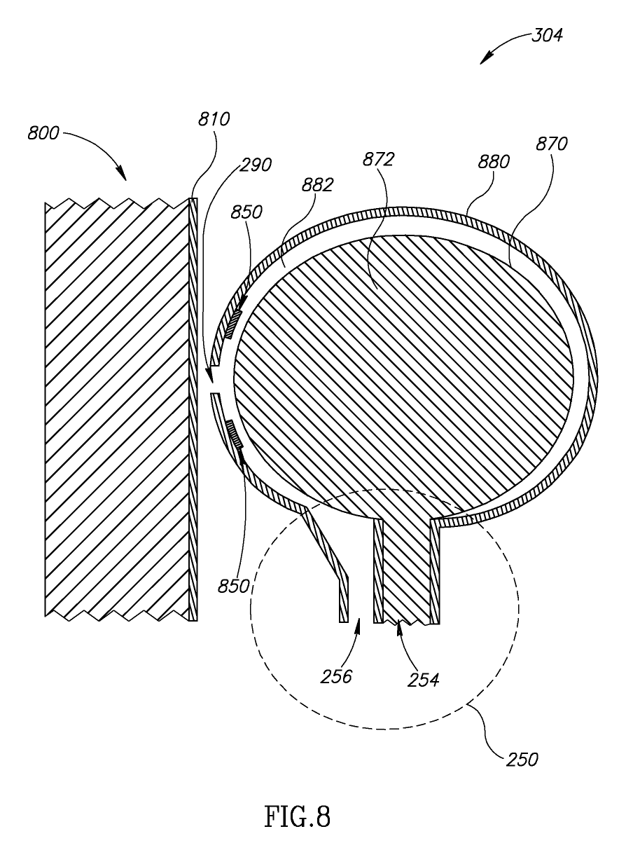

At 102 a pressure sensitive valve 300 is provided in a body of a subject. Optionally, provision 102 is by insertion along a guidewire 260. In an exemplary embodiment of the invention, an inner balloon 270 (see also FIG. 7) or 870 (FIG. 8), is inflated to a desired volume at a desired pressure, for example using a pump 210.

FIG. 1B describes the act of providing 102 in greater detail and will be explained below.

At 104 a pressure pulse is directed to valve 300 from a pressure source outside body, for example a pressure pulse “gun” 214.

The pressure pulse causes pressure in valve 300 to reach a desired injection pressure (Pᵢₙⱼ) and open 106. It is stressed that Pᵢₙⱼ can vary with a coefficient of elasticity “K” and/or a volume of one or more parts of valve 300 as will be explained below. As long as pressure within valve 300 remains at Pᵢₙⱼ or greater, fluid is expelled at a high velocity and valve 300 remains open 106.

In an exemplary embodiment of the invention, valve 300 maintains 108 an internal pressure of at least Pᵢₙⱼ until a sufficient volume of fluid has been ejected.

As fluid is ejected from valve 300, pressure in valve 300 drops 110 below Pᵢₙⱼ and valve 300 closes 112. A tendency of pressure in valve 300 to drop below Pᵢₙⱼ can be at least partially mitigated by supply of additional fluid to the valve at Pᵢₙⱼ or greater.

In the described embodiment, valve 300 is not damaged by being opened so that it can be opened 106 and closed 112 many times. In an exemplary embodiment of the invention, valve 300 can be reused. Optionally, re-use occurs at a same location or at a different location.

Depicted exemplary valve 300 comprises a pair of balloons (e.g. 270 and 280) nested one within the other.

In an exemplary embodiment of the invention, inner balloon 270 and outer balloon 280 are both elastic. Optionally, Pᵢₙⱼ according to this embodiment of valve 300 is governed at least in part by an inflation volume of inner balloon 270 and/or by an inflation pressure of inner balloon 270 and/or by K of outer balloon 280 at that volume.

In an exemplary embodiment of the invention, inner balloon 270 is elastic and outer balloon 280 is inelastic. Optionally, Pᵢₙⱼ according to this embodiment of the invention is governed at least in part by an available compliance volume of inner balloon 270. Compliance volume of inner balloon 270 may be affected by one or more of a compressibility of a material used to fill the balloon, an ability of a conduit connected to balloon 270 to accommodate fluid exiting the balloon, a compliant element in fluid communication with fluid in the system, a degree of compliance of pump 210 and a direction of flow of pump 210 at a relevant time.

In an exemplary embodiment of the invention, inner balloon 270 is inelastic but compliant and outer balloon 280 is elastic. Optionally, Pᵢₙⱼ according to this embodiment of valve 300 is governed at least in part by one or more of an inflation volume of inner balloon 270, by K of outer balloon 280 at that volume and by an available compliance volume of inner balloon 270 as described above.

According to various exemplary embodiments of the invention, medication injected from valve 300 reduces a likelihood of restenosis after a PTCA procedure and/or alters structural and/or electrical properties of tissue and/or delivers a therapeutic and/or cyto-toxic agent.

Referring now to FIGS. 1B and 2A, the act of providing 102 of valve 300 according to some exemplary embodiments of the invention is explained in greater detail.

In an exemplary embodiment of the invention, a medical procedure begins with insertion 118 of valve 300 comprising outer balloon 280 and inner balloon 270 into blood vessel 310 (see FIG. 3A). Optionally, insertion is along a guidewire 260. In an exemplary embodiment of the invention, valve 300 is used to perform a PTCA as well as to inject fluid so the insertion is to a site of stenosis 320.

After insertion 118 to a desired site, inner balloon 270 is inflated 120. If PTCA is to be performed, inflation can be to a PTCA pressure. A PTCA pressure is typically in excess of 5, 10, 20 or 30 atmospheres. In an exemplary embodiment of the invention, pressure for inflation is provided by a pump 210 which pumps fluid via tubing 216 and/or connector 220 to lumen 254 of catheter 250 which is in fluid communication with lumen 272 of inner balloon 270. In an exemplary embodiment of the invention, pressure supplied by pump 210 is monitored, for example by a gauge on pump 210 and/or by a pressure sensor in balloon 270. Optionally, initial inflation can be to a PTCA pressure and pressure can be reduced for subsequent operation of valve 300 as an injector.

In an exemplary embodiment of the invention, inner balloon 270 expands and contacts an inner surface of outer balloon 280 sealing 130 holes 290. Optionally, balloon 270 is expanded to a degree which concurrently opens holes 290 (e.g. by stretching) and seals holes 290 (e.g. by covering). In the embodiment depicted in FIG. 3B, balloon 270 is shown contacting holes 290. In other exemplary embodiments of the invention, balloon 270 prevents a flow of fluid to holes 290 by contacting portions of balloon 280 other than holes 290 (e.g. a ring at the neck of the balloon). Valve 300 is now in a closed operational state. If an optional PTCA is being performed, expansion of inner balloon 270 causes outer balloon 280 to expand 132 and open vessel 310.

In an exemplary embodiment of the invention, while valve 300 is closed, pump 212 delivers 140 liquid medication via lumen 256 of catheter 250 to an entrance to inner lumen 282 of outer balloon 280 at a pressure slightly Pᵢₙⱼ. Optionally, lumens 256 and/or 282 are pre-filled (e.g. with medication) prior to insertion 118. In an exemplary embodiment of the invention, pre-filling removes trapped air.

At this stage, inner balloon 270 continues 160 to seal holes 290 of outer balloon 280 so that valve 300 remains closed.

In order to cause valve 300 to open, a pulse gun 214 applies 170 a pressure pulse via lumen 256 so that pressure at the entrance to inner lumen 282 of outer balloon 280 increases to at least Pᵢₙⱼ. This increase causes fluid to flow into inner lumen 282 of outer balloon 280 at Pᵢₙⱼ. The pressure in lumen 282 of balloon 280 causes inner balloon 270 and outer balloon 280 to separate 180. Separation can result from contraction of inner balloon 270 (if it is sufficiently compliant) 180 and/or expansion of outer balloon 280 (if it is sufficiently elastic). In the depicted exemplary embodiment of the invention (FIG. 3D), contraction 180 of inner balloon 270 uncovers 182 at least some of holes 290 of outer balloon 280. Medication exits 184 via uncovered holes 290 at Pᵢₙⱼ or greater. In various exemplary embodiments of the invention, a degree to which an actual pressure driving exit 184 exceeds Pᵢₙⱼ can vary with a magnitude of the pressure pulse delivered by gun 214 and/or characteristics of catheter 250. Because the medication is driven by a relatively high pressure, it penetrates into tissue of blood vessel 310. The Pᵢₙⱼ at which valve 300 opens is optionally chosen according to a desired penetration profile of the medication.

FIGS. 3A to 3D, 3E to 3F and 7 and 8 illustrate exemplary pressure sensitive valves according to various exemplary embodiments of the invention which operate as described above and can be used in conjunction with a system as depicted in FIG. 2A.

### Operation of an Exemplary Pressure Sensitive Valve

FIGS. 3A, 3B, 3C and 3D illustrate operation of an exemplary nested balloon valve 300 graphically. Each of these figures is a lateral cross section of valve 300 showing a catheter 250, an optional guidewire 260, an inner structure 270 and an outer structure 280 with at least one hole 290 (a plurality of holes 290 are pictured) in a blood vessel 310, or other intrabody lumen. Optionally, pictured catheter 250 can be replaced by analogous fluid supply conduits. In the depicted valve 300, the outer structure comprising holes 290 is outer balloon 280 and the inner structure is inner balloon 270.

In this series of drawings a “rapid exchange” embodiment of catheter 250 is depicted. In the pictured embodiment, guidewire 260 is outside catheter 250 proximal to rapid exchange hole 262.

In other exemplary embodiments of the invention, catheter 250 is an “over the wire” catheter.

In other exemplary embodiments of the invention, catheter 250 is deployed without a guidewire. Deployment without a guidewire may be suitable, for example, in non-vascular applications. Non-vascular applications may include, for example, prostate treatment, urinary bladder treatment, rectal treatment, intranasal treatment, vaginal treatment and uterine treatment.

In FIG. 3A valve 300 is shown positioned in proximity to a stenosis 320 just prior to a PTCA procedure. This positioning of valve 300 is exemplary only.

FIG. 3B illustrates performance of PTCA using pump 210 (FIG. 2) to inject fluid via lumen 254 of catheter 250 into lumen 272 of inner balloon 270 (indicated by curved arrows) causing inner balloon 270 to expand 130. Expansion 130 pushes stenosis 320 outwards and opens blood vessel 310. Expansion 130 of inner balloon 270 closes holes 290 and brings them into close proximity with inner walls of vessel 310. Expansion 130 brings valve 300 to a closed operational state. In an exemplary embodiment of the invention, pump 210 inflates inner balloon 270 with a standard PTCA pressure, for example 5, 10, 12, 15, 17 or 20 atmospheres or intermediate or greater pressures. These pressures are generally sufficient to expand stenosis 320. Optionally, the pressure applied for PTCA contributes to defining Pᵢₙⱼ for operation of valve 300. Optionally, pressure in inner balloon 270 is adjusted after the PTCA procedure.

In an exemplary embodiment of the invention, Pᵢₙⱼ is defined by the following formula:

Pᵢₙⱼ=Pᵢₙₜₑᵣₙₐₗ−[Kᵢ−X]

Where: Pᵢₙₜₑᵣₙₐₗ is an inflation pressure of the inner balloon;
Kᵢ is a coefficient of elasticity of the inner balloon an
X is a constant.

The lower portion of FIG. 3B indicates a cross section of catheter 250 at three different (indicated) positions. These cross sections indicate how different lumens of catheter 250 can deliver fluid in an independently controllable manner to lumen 272 of inner balloon 270 and lumen 282 of outer balloon 280 and provide a conduit for guidewire 260 to pass through both balloons.

The left-most cross-section illustrates three lumens which characterize exemplary catheter 250 until it passes within inner lumen 282 of outer balloon 280. The three lumens are: an inner balloon catheter lumen 254, a guidewire catheter lumen 258 and an outer balloon catheter lumen 256. Outer balloon catheter lumen 256 ends in lumen 282 of outer balloon 280 where it delivers fluid. Valve 300 switches from a closed to an open state when fluid delivery via outer balloon catheter lumen 256 reaches Pᵢₙⱼ.

In an exemplary embodiment of the invention, outer balloon lumen 256 is ellipsoid, optionally elliptical, in cross section. A non-circular cross-sectional area of outer balloon lumen 256 contributes to a greater capacity to conduct a high pressure fluid pulse from pulse gun 214 to inner lumen 282 of outer balloon 280 by contributing to an increased fluid flow without changing an outer diameter of catheter 250.

In an exemplary embodiment of the invention, outer balloon lumen 256 is elliptical and has a major axis of 0.6 mm and a minor axis of 0.43 mm. This exemplary configuration for lumen 256 provides a cross-sectional area of 0.22 mm². In an exemplary embodiment of the invention, guidewire lumen 258 is characterized by an inner diameter of 0.38 mm and inner balloon lumen 254 is characterized by an inner diameter of 0.2 to 0.33 mm. This exemplary configuration permits the three lumens to be provided in a standard PTCA catheter with an outer diameter of 1.1 mm.

FIG. 2D shows another exemplary embodiment of outer balloon lumen 256 in a cross section at B-B. According to the depicted embodiment, outer balloon lumen 256 occupies a majority of the cross sectional area of catheter 250. Optionally, lumen 256 is at least partially concave in cross section. In an exemplary embodiment of the invention, having outer balloon lumen 256 occupy a majority of the cross sectional area of catheter 250 contributes to efficiency of conducting a pressure pulse from gun 214 to inner lumen 282 of outer balloon 280. Optionally, the contribution to efficiency of conducting a pressure pulse results from reduced friction of fluid against walls of lumen 256. Optionally, the contribution to efficiency of conducting a pressure pulse results from increased velocity of fluid within lumen 256. According to the depicted exemplary embodiment, lumen 256 is characterized by a cross-sectional area of 0.40 mm² and still fits within a standard PTCA catheter.

The middle cross-section in FIG. 3B illustrates two lumens which characterize exemplary catheter 250 within at least a portion of inner lumen 272 of inner balloon 270. The two lumens are: inner balloon lumen 254 and guidewire lumen 258. In FIG. 3B these two lumens are illustrated as being side by side in this portion of catheter 250. In another exemplary embodiment of the invention, these two lumens (258 and 254) are nested one within the other. Inner balloon lumen 254 ends in lumen 272 of inner balloon 270 where it delivers fluid to inflate inner balloon 270.

The right-most cross-section illustrates a single guide wire lumen 258 which characterizes exemplary catheter 250 from within inner lumen 272 of inner balloon 270 until a distal end of catheter 250.

FIG. 3C illustrates pumping 140 of medication into inner lumen 282 of outer balloon 280 at a pressure below Pᵢₙⱼ. Lumen 282 is external with respect to inner balloon 270. At this stage of operation of valve 300, medication is optionally pumped 140 by outer balloon pump 212 which can be, for example, a conventional PTCA pump. In an exemplary embodiment of the invention, pumps 212 and 210 are similar, optionally identical. In an exemplary embodiment of the invention, each of pumps 212 and 210 is equipped with a pressure gauge so that a user can deliver a desired pressure. Pump 212 pumps 140 medication into lumen 282 at a pressure below Pᵢₙⱼ (indicated by arrows emanating from lumen 256). The degree to which pressure delivered by pump 212 at this stage is below Pᵢₙⱼ can vary with an anticipated magnitude of a pressure pulse to be delivered later. Holes 290 remain closed by pressure in lumen 272 of inner balloon 270 at this stage.

Optionally, connector 220 is adjusted at this stage so that pulse gun 214 is connected to lumen 256 in place of pump 212. Adjustment may involve, for example, rotating a control lever of a stopcock or disconnecting pump 212 and connecting gun 214 in place thereof.

In an exemplary embodiment of the invention, both pump 212 and gun 214 are connected to lumen 256 concurrently. In an exemplary embodiment of the invention, an output from gun 214 enters lumen 256 downstream of an output from pump 212.

FIG. 3D shows delivery of a pulse of pressure (represented as arrows) from pulse gun 212 via lumen 256 of catheter 250 into lumen 282 of outer balloon 280. Delivery of the pulse raises pressure 170 in lumen 282 at least to Pᵢₙⱼ, optionally to a pressure well in excess of Pᵢₙⱼ As soon as pressure in lumen 282 reaches Pᵢₙⱼ (e.g. by exceeding T), holes 290 are uncovered 182 and medication exits holes 290 at high velocity.

In the depicted embodiment, holes 290 may become uncovered 182 because inner balloon 270 contracts and/or because outer balloon 280 expands. The extent to which inner balloon 270 contracts and/or outer balloon 270 expands may be influenced by one or more of elasticity of inner balloon 270, elasticity of outer balloon 280, a magnitude of a difference between pressure in lumen 272 of inner balloon 270 and Pᵢₙⱼ, opposing forces applied by vessel 310, compliance of lumen 256, compliance of gun 214 and compliance of pump 212.

In an exemplary embodiment of the invention, pressure in lumen 272 of inner balloon 270 is optionally 8, 10, 12, 14 or 16 atmospheres or lesser or greater or intermediate pressures, which is typically sufficient to open a stenosis.

In an exemplary embodiment of the invention, a pressure pulse wave of 100 to 280 atmospheres exiting pulse gun 214 produces an initial velocity of fluid in lumen 256 of 20, 50, 75 or 100 meters/second or lesser or intermediate or greater values. A pulse wave of this magnitude exiting gun 214 provides at least Pᵢₙⱼ in lumen 282 of valve 300 and switches the valve from closed to open.

A magnitude of the pulse delivered by gun 214 can be controlled by manipulating force applied by an actuation mechanism (e.g. gas pressure or spring resistance) in gun 214.

As the pulse wave moves through lumen 256 of catheter 250, the pressure pulse wave is reduced in amplitude. A degree of amplitude damping can vary with length and/or cross-sectional area of lumen 256 and materials employed in catheter construction.

When a leading edge of the wave reaches lumen 282 of outer balloon 280, pressure in lumen 272 of inner balloon 270 tends to prevent the leading edge of the pressure wave from proceeding further. As more of the wave arrives, pressure to enter lumen 282 increases. In an exemplary embodiment of the invention, when the pressure reaches Pᵢₙⱼ minus 2 atmospheres, fluid begins to enter lumen 282. A pressure at which fluid begins to enter lumen 282 may vary with one or more pressure in lumen 272 of inner balloon 270, elastic properties of outer balloon 280 and a counter-expansive force applied to balloon 280 by vessel 310. When pressure in lumen 282 reaches Pᵢₙⱼ holes 290 open and fluid is ejected at high velocity. In an exemplary embodiment of the invention fluid is ejected from valve 300 at 14, optionally 20, optionally 30, optionally 34, optionally 40 atmospheres or intermediate or greater values. In an exemplary embodiment of the invention fluid is ejected from valve 300 at an average velocity greater than 10, optionally 20, optionally 50, optionally 100, optionally 200 m/s. In some preferred embodiments of the invention, increasing ejection velocity and/or injection pressure contributes to a greater depth of penetration and/or a shorter injection time.

Optionally, the pressure wave continues to arrive in lumen 282 after holes 290 open. In an exemplary embodiment of the invention, actual pressure in lumen 282 during an injection event exceeds pressure in lumen 272 of inner balloon 270 by 2, optionally 4, optionally 8, optionally 16, optionally 24 atmospheres or intermediate or greater pressure differentials.

In an exemplary embodiment of the invention, a degree by which Pᵢₙⱼ must exceed pressure in lumen 272 of inner balloon 270 and/or an actual pressure desired in lumen 282 during an injection event is considered when planning a pressure pulse to deliver fluids. If Pᵢₙⱼ and/or an actual pressure in lumen 282 during an injection event exceeds pressure in lumen 272 of inner balloon 270 by too much, an exit velocity of fluid from holes 290 can be excessive. Excessive exit velocity can potentially cause tissue damage and/or cause delivery of fluid to an incorrect tissue layer and/or damage outer balloon 280 and/or inner balloon 270.

Optionally, ejection of fluid from holes 290 lasts 5, 10, 20, 50, 75 or 100 milliseconds or lesser or intermediate or greater times. During this ejection time, pressure in lumen 282 remains at least at Pᵢₙⱼ, and may optionally be much higher. Optionally, a degree by which pressure in lumen 282 of valve 300 differs from pressure in lumen 272 of inner balloon 270 can remain constant or vary during this time interval. In an exemplary embodiment of the invention, the degree by which pressure in lumen 282 of valve 300 differs from pressure in lumen 272 of inner balloon 270 increases and then decreases during this time until pressure in lumen 282 falls below Pᵢₙⱼ. Delivery of the pressure wave is described in greater detail below in a section entitled “Pulse Wave Delivery”.

Optionally, pump 212 and gun 214 are incorporated into a single apparatus.

When valve 300 opens as a result of an applied pressure pulse, exit 184 of medication causes pressure in lumen 282 of outer balloon 280 to return 190 to drop below Pᵢₙⱼ so that inner balloon 270 covers holes 290 closing valve 300.

Optionally, this sequence of opening/closing valve 300 can be repeated cyclically at a same location or a series of different locations. According to various exemplary embodiments of the invention, valve 300 can deliver multiple doses of medication to a single site (e.g. stenosis site) by application of multiple pressure pulses from gun 214. Alternatively or additionally, valve 300 can deliver medication to multiple sites if it is navigated to additional sites between pressure pulses from gun 214.

In other exemplary embodiments of the invention a pressure of at least Pᵢₙⱼ is applied as a constant pressure (as opposed to a pulse) and valve 300 remains open until application of the pressure ceases.

In an exemplary embodiment of the invention, operation of valve 300 causes ejection of medication from at least 50, 60, 70, 80, 85, 90, 95 or substantially 100% of holes 290.

### Additional Exemplary Valve Configurations

FIGS. 7 and 8 are cross sectional drawings of additional exemplary valve configurations 302 and 304 respectively according to embodiments of the invention.

FIG. 7 illustrates a non-cylindrical valve 302 with deflated inner balloon 270D (solid line) and inflated inner balloon 270I (dotted lines). As described above with reference to FIGS. 3A, 3B, 3C and 3D, introducing liquid or gas via lumen 254 of catheter 250 into inner lumen 272 of balloon 270 inflates inner balloon 270 so that it expands and inflates outer balloon 280. Holes 290 in outer balloon 280 are covered by inner balloon 270.

Subsequent introduction of medication via lumen 256 of catheter 250 to inner lumen 282 of outer balloon 280 creates a separative force between the two balloons. In an exemplary embodiment of the invention, delivery of a pressure pulse via lumen 256 of catheter 250 causes the separative force to reach and/or exceed Pᵢₙⱼ.

When Pᵢₙⱼ is reached or exceeded, balloons 270 and 280 separate and medication flows through lumen 282 and outward from holes 290. Holes 290 are pictured in FIG. 7 as being distributed throughout the surface of outer balloon 280. This exemplary arrangement of holes produces radial ejection of fluid with respect to balloon 280.

However, in other exemplary embodiments of valves according to the invention, holes 290 may be concentrated in a particular area of balloon 280 to achieve ejection of medication in a desired direction.

Non-cylindrical valves 302 may optionally be useful in non-tubular lumens. Non-tubular lumens include nostrils and nasal sinuses.

FIG. 8 illustrates a valve 304 with an outer structure 880 constructed of an inelastic material, optionally with a shape memory. In the depicted embodiment, a single hole 290 is pictured. In FIG. 8, inner balloon 870 is shown only partially inflated so that hole 290 is unblocked and valve 304 is open.

In other exemplary embodiments of the invention, multiple holes 290 are provided on outer structure 880. In an exemplary embodiment of the invention, placement of hole or holes 290 is used to choose one or more ejection directions. Optionally, one or more markers 850 (e.g. radio-opaque markers) are provided on outer structure 880. In an exemplary embodiment of the invention, markers 850 and a surface 810 of a target tissue 800 are visualized by medical imaging (e.g. X-ray or fluoroscopy). Optionally, outer structure 880 can be rotated or otherwise adjusted to bring markers 850 into a desired orientation with respect to target tissue 800 so that ejection of medication from hole(s) 290 will be into a desired location on surface 810 of target tissue 800. Ejection of fluid is accomplished by delivery of a pressure pulse to achieve Pᵢₙⱼ or greater as described above.

In an exemplary embodiment of the invention, valve 304 is used to deliver a single high speed jet of fluid to a specific sight inside the body, optionally from a single hole 290.

Referring again to FIG. 3D, in some exemplary embodiments of the invention only one balloon is employed to form valve 300. Optionally, an elastic outer balloon 280 with holes 290 is filled with an inelastic form 270 of sufficient strength to resist deformation at Pᵢₙⱼ or other pressures in lumen 282 which may result from delivery of a pressure pulse as described above. Operation of this exemplary valve 300 is similar to that described above except that Pᵢₙⱼ is used to overcome elastic contraction of outer balloon 280 against inelastic form 270. Optionally, inelastic form 270 is provided as a solid body. A coefficient of elasticity K of outer balloon 280 at a volume defined by inner structure 270 substantially governs Pᵢₙⱼ according to this embodiment of the invention. Briefly, delivery of PP from gun 214 causes pressure in lumen 282 of outer balloon 280 to reach and/or exceed Pᵢₙⱼ. Inelastic form 270 does not contract at Pᵢₙⱼ. According to this exemplary embodiment, shape and/or volume of inner structure 270 contribute to Pᵢₙⱼ. Outer balloon 280 expands, opening holes 290 and permitting ejection of fluid therefrom. This type of valve configuration can be useful in applications where an outer diameter of valve 300 is not a limit for providing 102 and/or insertion 118.

FIGS. 3E and 3F depict another exemplary valve configuration 310 in lateral cross section (FIG. 3E) and transverse cross section at line C-C (FIG. 3F). In valve 310, inner structure 270, depicted as a balloon 270 with a lumen 272, is provided with ribs 370 on its external surface. Ribs 370 contact outer balloon 280 and divide lumen 282 into a plurality of flow channels 382. According to this exemplary embodiment of the invention, a flow of fluid into channels 382 is blocked below Pᵢₙⱼ by one or more microvalves 390 which are each individually set to open at Pᵢₙⱼ. Microvalves 390 can be provide as rupture discs, snap-valves or spring actuated valves. Valves 310 containing microvalves 390 configured as rupture discs or snap valves are single use valves. Valves 310 containing microvalves 390 configured as spring actuated valves can be re-usable. In an exemplary embodiment of the invention, when microvalves 390 open at Pᵢₙⱼ, fluid rushes into channels 382 of lumen 282 of outer balloon 280 and accelerates towards and through holes 290.

In an exemplary embodiment of the invention, the configuration depicted in FIG. 3E can be applied to a solid inner body 270 at least partially covered by an outer membrane 280 to define channels 282.

Exemplary Hole Geometries

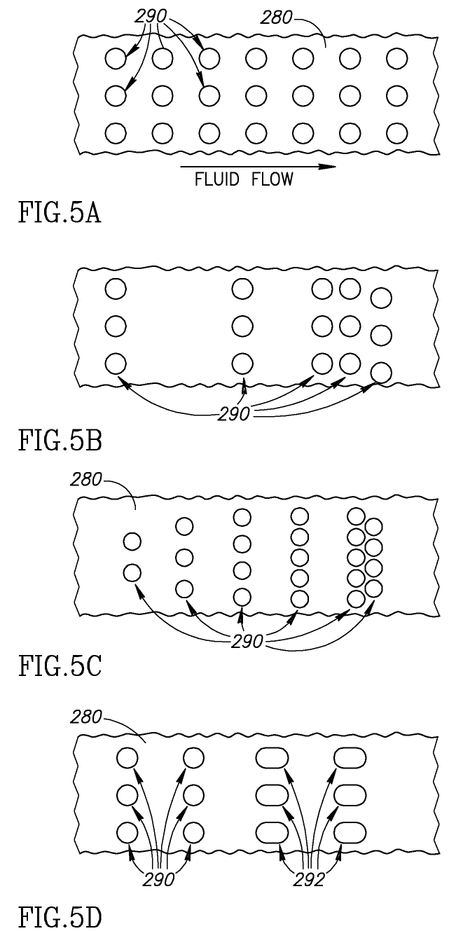

FIGS. 5A, 5B, 5C and 5D illustrate exemplary patterns of holes 290 on outer balloon 280. In all of these figures, balloon 280, which is typically cylindrical or ovoid is depicted in a cut plan view. Vertical rows of holes 290 represent circumferential rings about balloon 280. Flow of medication under pressure into lumen 282 is from left to right in all 4 drawings as indicated by the arrow in FIG. 5A. Holes 290 are optionally round or elliptical or slits.

In an exemplary embodiment of the invention, round holes 290 have a diameter of 10, 20, 30 or 40 microns or lesser or intermediate or greater diameters. Optionally, as dimensions of a hole 290 increase, an ability of the hole to dissipate pressure increases. However, if holes 290 are too large (e.g. diameter of 50 μm more in), injection may occur only through those holes 290 located in a proximal portion of balloon 280. Conversely, if holes 290 are too small (e.g. diameter of 1-5 μm) the desired high velocity ejection of fluid may be replaced by sweating or dripping of fluid from balloon 280.

FIG. 5A depicts an exemplary embodiment in which holes 290 are arranged in equally spaced vertical rows. Optionally, distance between holes 290 in a row is equivalent. This exemplary arrangement allows pressure in lumen 282 to decrease as the pressure wave moves from left to right because each additional row of holes releases pressure from lumen 282. In some exemplary embodiments of the invention, a non-constant pressure in lumen 282 is not desired.

FIG. 5B depicts an exemplary embodiment in which holes 290 are arranged in vertical rows with a decreasing distance between each successive row. Optionally, distance between holes in a row is equivalent. This configuration is designed to contribute to equalization of an amount of medication delivered per unit length of balloon 280 by providing additional holes in a distal portion of balloon 280 where pressure in lumen 282 is lower. Optionally, penetration depth is lower in a distal portion of balloon 280 in this configuration.

FIG. 5C depicts an exemplary embodiment in which holes 290 are arranged in vertical rows with an increasing number of holes per row. Optionally, distance between rows is equivalent. This configuration is designed to contribute to equalization of an amount of medication delivered per unit length of balloon 280. Optionally, equalization is achieved by reducing a degree by which pressure in lumen 282 is lowered in a proximal portion of balloon 280.

FIG. 5D depicts an exemplary embodiment in which proximal holes 290 are of a smaller size and distal holes 292 are of a larger size. Optionally, distance between vertical rows is equivalent. Optionally, distance between holes within a row is equivalent. This configuration is designed to contribute to equalization of an amount of medication delivered per unit length of balloon 280 by providing additional cross sectional area of holes in a distal portion of balloon 280 where a difference between T and pressure in lumen 282 is lower.

In another exemplary embodiment of the invention (not pictured) a diameter of holes 290 increases incrementally and distally along an axis of balloon 280. This exemplary embodiment is designed to contribute to equalization of an amount of medication delivered per unit length of balloon 280 as for the embodiments depicted in FIGS. 5B, 5C and 5D. Embodiments of this type can offer an advantage in manufacturing as production of a relatively small number of larger holes with larger intervening spaces may be less difficult than production of a relatively large number of smaller holes with smaller intervening spaces.

The exemplary embodiments depicted in FIGS. 5A, 5B, 5C and 5D are all designed to provide radially symmetric ejection of medication with respect to a long axis of outer balloon 280. However, in some exemplary embodiments of the injection, non-radially symmetric ejection is provided. Non radially symmetric ejection may be achieved, for example, by providing holes 290 and/or 292 only on a desired angular range of balloon 280 with respect to its long axis. For example, holes 290 and/or 292 can be provided on 180, 120, 90, 60, 45 or 30 degree circumferential arcs of balloon 280 or lesser or intermediate or greater circumferential arcs of balloon 280. Non-radially symmetric hole configurations may be useful in treating lumen abnormalities which occur only on a selected portion of a lumen circumference. Because a delivered medication may be harmful to normal tissue, restriction of delivery to an actual abnormal target can be advantageous. For example, delivery of cyto-toxic material on one face of a lumen can be medically desirable while delivery of the same material to normal tissue on an opposite side of the lumen can be detrimental.

In other exemplary embodiments the non radially symmetric distribution of holes 290 be used to inject two or more times at different circumferential portions of a lumen. Desired circumferential portions of the lumen can be selected by rotating valve 300 between ejection events. In an exemplary embodiment of the invention, multiple ejection events according to this strategy contribute to a more homogeneous delivery of medication throughout the target.

In other exemplary embodiments of the invention, balloon 280 is non-cylindrical. Non cylindrical balloons 280 can optionally be symmetric or non symmetric. Holes 290 and/or 292 can be provided on any desired portion of balloon 280 to provided ejection of medication in a desired direction. Design and potential clinical applications of exemplary non-cylindrical balloons are described in co-pending U.S. patent application 2006/0190022 which is fully incorporated herein by reference. Additional exemplary balloon configurations are described below in.

In an exemplary embodiment of the invention, markers are provided on balloon 280 to aid in orientation of balloon 280 within the body so that ejection of medication in a desired direction can be achieved. Optionally, the markers are radio-opaque markers 850 (FIG. 8) so that they can be detected in X-ray or fluoroscopy images.

According to exemplary embodiments of the invention, acceleration of fluid can occur in lumen 282 and/or in holes 290 and/or after exiting holes 290.

Optionally, holes 290 are configured as truncated cones. For example each hole 290 can have a diameter of 20μ at an inner surface of balloon 280 and a diameter of 25μ at the outer surface of balloon 280. Due to mass conservation (V*A=V*A) the velocity of the fluid decreases while flowing in the channel. Optionally, the velocity of the fluid decreases in the hole and the fluid accelerates according to Bernoulli's principle when leaving the channel where the pressure is reduced to substantially zero.

Exemplary Ejection Control Mechanism

As described above, pressure in lumen 282 tends to decrease in distal portions of a cylindrical balloon 280 due to release of pressure from holes 290 in a proximal portion of the balloon. This can contribute to reduced ejection velocity and/or volume from holes located in a distal portion of balloon 280. However, it is possible to inject medication several times from the same balloon 280.

In an exemplary embodiment of the invention, an axially translatable sleeve is provided between balloon 280 and vessel 310. Optionally, the sleeve is positioned so that it does not cover any of holes 290 and/or 292 in an initial operational cycle of valve 300. With each subsequent operational cycle, the sleeve is moved axially distally so that an increasing portion of proximal holes 290 and/or 292 are covered. In an exemplary embodiment of the invention, pressure T in inner balloon 270 insures contact between outer balloon 280 and the sleeve. Optionally, a pressure in inner balloon 270 can be reduced to make it easier to advance the sleeve along outer balloon 280 within vessel 310.

Use of the sleeve to cover a subset of holes 290 and/or 292 can reduce dissipation of pressure in lumen 282 in a proximal portion of balloon 280, wherein the proximal portion increases with each successive operational cycle.

In other exemplary embodiments of the invention only one ring of holes 290 is provided on balloon 280. According to these embodiments of the invention, valve 300 is opened once as described above to provide an initial injection into a portion of a target. Valve 300 can then be repositioned one or more times and re-opened to inject into additional portions of the target. In an exemplary embodiment of the invention, the one ring of holes 290 is positioned on a distal portion of balloon 280. Optionally, a series of injections into a site of former stenosis 320 are performed as valve 300 is being withdrawn after PTCA.

In other exemplary embodiments of the invention a sleeve with one or more openings is provided. The openings can be configured to include a desired subset of holes 290. Optionally, axially and/or rotational translation of the sleeve with respect to outer balloon 280 between injections can be used to sequentially eject medication from different subsets of holes 290. Pulse wave delivery.

Delivery of a pressure pulse to provide Pᵢₙⱼ in lumen 282 of balloon 280 can be achieved using a wide variety of pressure sources.

Exemplary pulse guns suited for use in the context of exemplary embodiments of the invention can be found in the field of needless injectors where the injection is performed through one orifice of about 100 μm diameter. For example, U.S. Pat. No. 5,730,723 (the disclosure of which is fully incorporated herein by reference) is an example of a gas powered gun and U.S. Pat. No. 5,704,911 (the disclosure of which is fully incorporated herein by reference) is an example of a spring loaded gun. In an exemplary embodiment of the invention, the single 100 μm hole of the needless injectors described in these earlier patents is replace by holes 290 of balloon 280. Optionally, a total cross sectional area of holes 290 is 0.04, 0.5, 1, 2, 3, 4 or 5 mm or lesser or greater or intermediate areas.

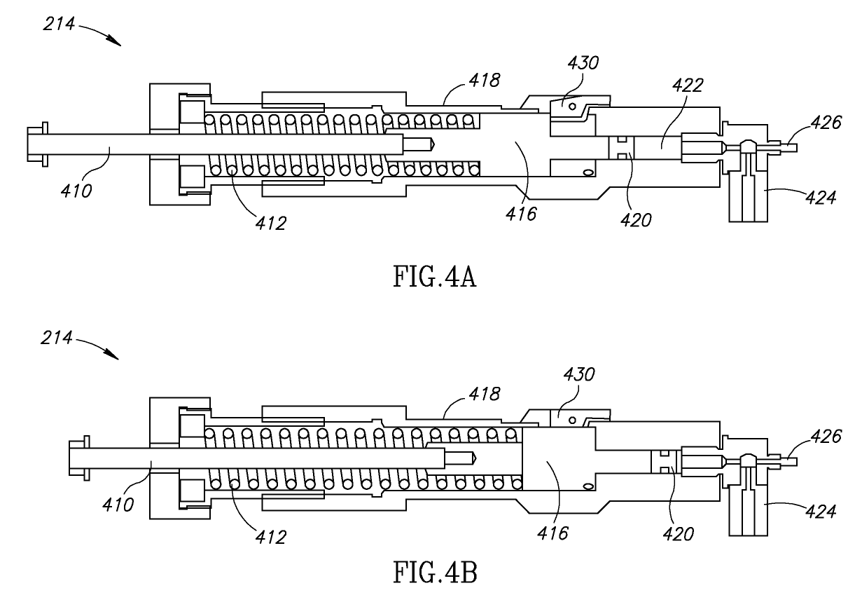

FIGS. 4A and 4B are lateral cross sectional views of a spring activated pulse gun 214 according to an exemplary embodiment of the invention in “cocked” and “fired” states respectively. The depicted exemplary pulse gun comprises a housing 418, a screw handle 410 to load the gun, a spring 412, a piston 416, a floating piston 420, a medication reservoir 422, a fill connector 424 and, an optional trigger 430 an exit port 426.

As seen in FIG. 2A, exit port 426 is optionally connected to tubing 216 which is, in turn, connected to lumen 256 of catheter 250. In this way, fluid exiting port 426 can be routed to lumen 282 of outer balloon 280. In different exemplary embodiments of the invention, an optional fill connector 424 is attached to a medication vial (not shown) or to tubing 216 attached to an output from pump 212. Optional fill connector 424 permits an inflow of medication to reservoir 422 when gun 214 is cocked. Optionally, a single gun 214 can deliver more than one medication during a treatment, for example by stepping release of piston 416 by trigger 430.

FIG. 4A illustrates cocking of gun 214 to store energy as compression in spring 412 and fill medication reservoir 422 with medication. Withdrawal of handle 410 against resistive force of spring 412 increases a volume of medication reservoir 422 by causing floating piston 420 to move towards handle 410. In an exemplary embodiment of the invention, motion of piston 420 draws medication into reservoir 422 from fill connector 424. Connector 424 is optionally equipped with a directional pressure sensitive valve and/or stopcock so that medication is directed to exit port 426 when gun 214 is fired. Optionally, trigger 430 locks handle 410 in an extended position by engaging piston 416 so that gun 214 can easily be maintained in a “cocked” operative state.

FIG. 4B illustrates firing of gun 214. Operation of optional trigger 430, or release of handle 410 by other means, allows force stored in spring 412 to propel piston 416 forward. Forward motion of piston 416 drives floating piston 420 forwards and reduce a volume of medication reservoir 422. This reduction in volume creates a sudden increase in pressure in medication reservoir 422. In an exemplary embodiment of the invention, floating piston 420 traverses medication reservoir 422 and effectively reduces a volume thereof to zero. A resultant pressure pulse propels medication from reservoir 422 outwards through exit port 426 and through lumen 256 of catheter 250 as described above. Optionally, floating piston 420 is equipped with on O-ring or similar seal to reduce unwanted leaking of medication from medication reservoir 422

Optionally, lumen 256 has a cross-sectional area of 0.22-0.4 mm² and a length of 1000 mm so that a total volume of lumen 256 is about 0.22-0.4 cc. Optionally, an aliquot of medication in reservoir 422 has a volume of 0.05 to 0.2 cc, optionally about 0.1 cc. In an exemplary embodiment of the invention, delivery of a single pulse from gun 214 causes a 50 to 90 percent increase in pressure in lumen 256. This pressure causes holes 290 to open which dissipates the added pressure as described above by permitting medication to exit holes 290. Optionally, flow of the medication in lumen 256 continues even when pressure is dissipated by ejection of fluid from holes 290 due to continued movement of floating piston 420.

### Exemplary Pulse Wave Amplification

In an exemplary embodiment of the invention, delivery of a pressure pulse to provide Pᵢₙⱼ in lumen 282 of balloon 280 is timed to coincide with a withdrawal of a small volume of fluid from lumen 272 of inner balloon 270. Withdrawal of a small volume of fluid from lumen 272 of inner balloon 270 can be accomplished, for example, by reversing a flow direction of pump 210 for a short period of time. In an exemplary embodiment of the invention, withdrawal of a small volume of fluid from lumen 272 of inner balloon 270 imparts compliance to balloon 270 and/or increases an available volume of inner lumen 282 of outer balloon 280 to a small degree. Optionally, one or more of these effects reduce Pᵢₙⱼ slightly so that an effect of the pulse wave delivered by gun 214 is amplified.

### Exemplary Conduit Construction

FIG. 2A shows a system of conduits which conduct fluids from pumps 210 and 212 and from gun 214 to valve 300. In the depicted embodiment, tubing 216 from pumps 210 and 212 and from gun 214 converges at connector 220. Between connector 220 and catheter 250, a conduit 226 conducts fluid destined to inner lumen 282 of outer balloon 280 and fluid destined to inner lumen 272 of inner balloon 270.

FIG. 2C depicts an exemplary embodiment of conduit 226 in a cross section at A-A. In the depicted embodiment, conduit lumen 256 is nested within conduit lumen 254. Optionally, an outer wall of lumen 254 is constructed of Nylon or a stronger material such as, for example, PEEK and an outer wall of lumen 256 is constructed of SS 304. In an exemplary embodiment of the invention, conduit 226 is 600, optionally 800, optionally 1000 mm long or lesser or intermediate or greater lengths. In an exemplary embodiment of the invention, a shorter conduit 226 contributes to a reduction in dissipation of a pressure pulse emanating from gun 214.

FIG. 2B is a cross section of catheter 250 at B-B illustrating catheter lumens 254 and 256 which are extensions of similarly numbered conduit lumens. Optionally, catheter 250 includes a third lumen 252 for guidewire 260. In FIG. 2B the three lumens of catheter 250 are depicted in an exemplary parallel non-concentric configuration. In other exemplary embodiments of the invention, the lumens can be arranged concentrically.

FIG. 2D shows an alternate exemplary parallel non-concentric configuration of the three catheter lumens at B-B. In the pictured embodiment lumen 256 is increased in cross sectional area. Optionally, providing at least a portion of the outline of lumen 256 as a concave curve contributes to the increase in cross sectional area.

### Exemplary Construction Considerations

Optionally, pump 210 and/or pump 212 are standard PTCA pumps such as, for example those produced by Johnson and Johnson (e.g. deflator MX1380LB) or Medtronics (e.g. indeflator AC2200 Minneapolis, Minn.; USA).

In an exemplary embodiment of the invention, tubing 216 is hypo tubing, for example of the type manufactured by Creganna Medical Devices (Galway; Ireland; UK) In an exemplary embodiment of the invention, outer balloon 280 is constructed of an elastic material such as for example, nylon. Optionally, the nylon is 15, 20, 25, 30, 35, or 40 μm thick. Nylon suitable for use in construction of balloons 280 may be purchased, for example, from Polymerex Medical Corp (San Diego, Calif., USA).

Optionally, increasing thickness of the nylon used to construct outer balloon 280 increase strength of the balloon and/or reduces elasticity thereof.

In various exemplary embodiments of the invention, inner balloon 270 can be constructed of an elastic material or an inelastic material.

Suitable elastic materials for construction of balloon 270 include, but are not limited to nylon as described above for outer balloon 280.

Suitable inelastic (relative to Nylon) materials for construction of balloon 270 include, but are not limited to PET such as that manufactured by Advance Polymer (Salem, N.H., USA).

Optionally, an elastic inner balloon 270 “snaps back” as pressure in lumen 282 decreases from Pᵢₙⱼ (or greater) to T and then below T. In an exemplary embodiment of the invention, the “snapping back” can cause additional ejection of medication from holes 290. In an exemplary embodiment of the invention, energy provided by “snapping back” can substitute for a portion of the energy pulse provided by gun 214. In an exemplary embodiment of the invention, “snapping back” occurs rapidly enough to become part of the ejection of medication, which optionally persists 5 to 100 milliseconds.

In an exemplary embodiment of the invention, holes 290 in balloon 280 are prepared by micro-drilling. Micro-drilling equipment is available, for example, from Spectralytics (South Dassel, Minn.; USA).

Exemplary catheters 250 of the type described above may be manufactured, for example, by Minnesota MedTec (Minneapolis, Minn.; USA).

### Exemplary Medical Protocols

Optionally, valves according to the invention may be sized for specific applications. For example, in some exemplary embodiments of the invention, a valve for coronary applications might have a diameter of 2 to 3.5 mm and a length of 10 to 25 mm.

According to other exemplary embodiments of the invention, a valve intended for deployment in the prostate might be considerably larger, for example a diameter of 6 to 11 mm and a length of 20 to 40 mm.

Alternatively or additionally, particular modifications may be desired for certain vessel types. For example, the aorta is thicker, while a coronary vessel is thinner, thus suggesting different ejection parameters, powers and/or Pᵢₙⱼ and sizes. For example, an aorta may be 3 mm thick, while a coronary vessel may be less than 1 mm thick.

Exemplary Injection Results

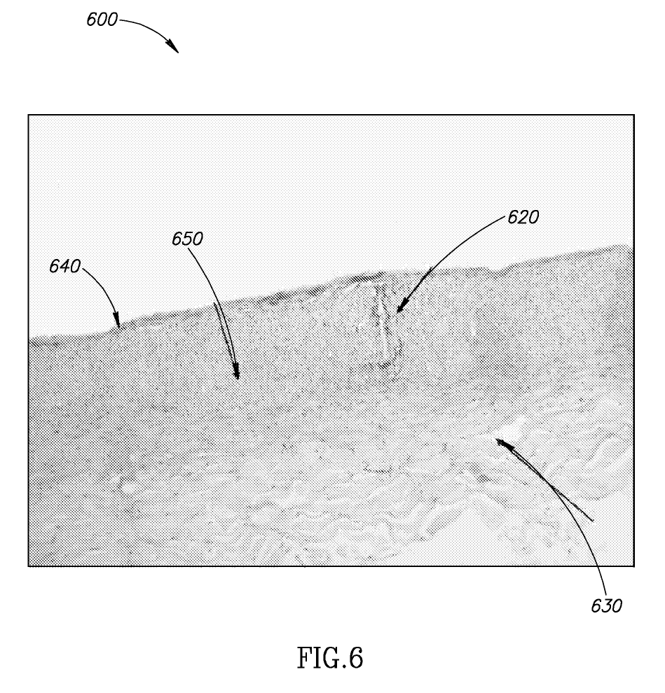

FIG. 6 is a micrograph 600 illustrating exemplary injection results from injection of a solution including black dye into an arterial wall using a valve 300 according to an exemplary embodiment of the invention. Micrograph 600 is a representative field of view of tissue injected with a valve 300 including an outer balloon 280 with 128 holes 290 characterized by a 20 μm diameter. A 120 atmosphere pulse pressure (PP) was provided by a gun 214 of the type depicted in FIG. 4A and described above. A volume of 0.1 cc was ejected from gun 214. The inner balloon 270 was inflated with 12 atmospheres of pressure. Under these conditions Pᵢₙⱼ is estimated to be in the range of 16-25 atmospheres following delivery of the pressure pulse from gun 214.

After injection the artery was removed, fixed in 4% paraformaldehyde and embedded in paraffin. A microtome was used to cut 4 μm sections which were mounted on glass slides and de-parafinized and stained with Haemotoxylin/Eosin using standard protocols. Black dye (seen most clearly at 620) from the injection penetrated intimae 640 and arrived deep within media 650 but did not reach adventitia 630 of the arterial wall.

Measurements are provided to serve only as exemplary measurements for particular cases. The exact measurements stated in the text may vary depending on the application, the type of vessel (e.g., artery, vein, xenograft, synthetic graft), shape of plaque (e.g., local, elongate, thin, thick, outer remolding, vulnerable) and/or sizes of vessels involved (e.g., 1 mm, 2 mm, 3 mm, 5 mm, aorta sized).

A wide variety of medications may be injected by apparatus or methods according to exemplary embodiments of the invention. Medications can include, but are not limited to, structural materials, anti-clotting agents, anti-cell proliferation agents, cytotoxic materials (e.g. chemotherapeutic agents, organic solvents (e.g. alcohols), fibrotic agents and metals (e.g. gold). Fibrotic agents may include, but are not limited to, formalin, papavain and curarc.

In an exemplary embodiment of the invention, induction of fibrosis in the target tissue can block an electrical signal. Blocking of an electric signal can contribute to regulation of cardiac rhythm.

In an exemplary embodiment of the invention, cytotoxicity is desirable, for example in tumor treatment or other targeted tissue ablation. Targeted tissue ablation may have applications, for example, in treatment of atrial fibrillation and/or to mimic the effects of intestinal resection.

### Exemplary Pressure Profile

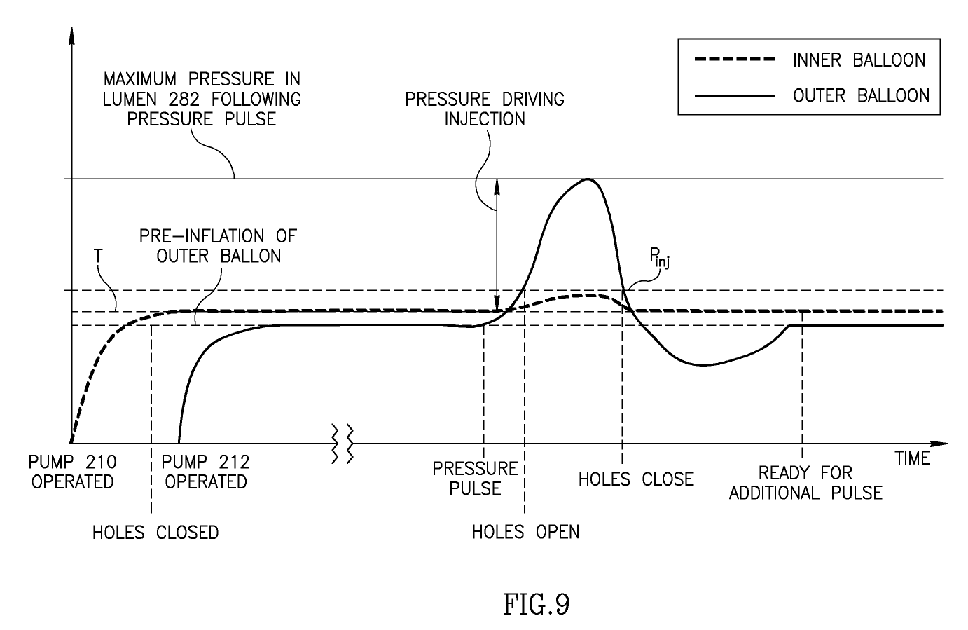

FIG. 9 is a graph illustrating pressure in outer balloon 280 (solid line) and inner balloon 270 (dashed line) of an exemplary valve 300 of the general configuration described above as a function of time prior to, during, and after an exemplary injection event.

In an exemplary embodiment of the invention, after valve 300 is positioned at a desired location pump 210 is operated and inner balloon 270 is inflated to T (e.g. 10 atmospheres). In the depicted embodiment, all holes 290 are closed at this stage. Pump 212 is then operated to bring a pressure in lumen 282 of balloon 280 to a pre-inflation pressure slightly below T (e.g. 8 atmospheres).

In an exemplary embodiment of the invention, a subsequent pressure pulse causes pressure in lumen 282 to exceed Pᵢₙⱼ. At least some of holes 290 open at this stage. Optionally, pressure in lumen 272 of inner balloon 270 also rises slightly as pressure in lumen 282 of outer balloon 280 causes inner balloon 270 to contract.

In the depicted embodiment, the delivered pulse continues to increase pressure in lumen 282. Optionally, pressure in lumen 282 may exceed T by 4, 8, 12, 16 or 20 atmospheres or lesser or greater or intermediate pressure differentials. The pressure differential drives injection of liquid medication from holes 290 into surrounding tissue.

After the pulse, pressure in lumen 282 begins to decrease and eventually drops below Pᵢₙⱼ , at which point holes 290 close. In the depicted embodiment, pressure in lumen 282 of outer balloon 280 drops momentarily below the pre-inflation pressure. Optionally, pump 212 brings pressure in lumen 282 of outer balloon 280 back to the pre-inflation pressure and valve 300 is ready to receive an additional pulse.

### Exemplary use in Conjunction with Atherectomy

In an exemplary embodiment of the invention, valve 300 (or 302 or 304 or other exemplary configurations) is used to deliver medication to an atherectomy site during or shortly after performance of the atherectomy.

Atherectomy may be performed, for example, using commercially available devices.

One commercially available atherectomy device is a Rotoblator (Heart Technology Inc., Bellevue, Wash., USA). Rotoblator type devices and Atherectomy procedures using same are described in, for example, U.S. Pat. Nos. 4,990,134; 5,314,407 and 5,364,393, the disclosures

Another commercially available atherectomy device is a “SilverHawk™” (Fox Hollow Technologies Inc., Menlo Park, Calif., USA). SilverHawk type devices and Atherectomy procedures using same are described in, for example, U.S. Pat. Nos. 6,027,514; 6,447,525; 6,629,953 and 6,638,233 the disclosures of which are fully incorporated herein by reference.

As in PTCA, atherectomy sites are prone to restenosis and/or arterial collapse. Delivery of appropriate medications as described above for PTCA is potentially beneficial in the context of an atherectomy procedure.

Atherectomy catheters that include imaging capabilities are described at least in U.S. Pat. Nos. 6,299,622; 6,623,496 and 6,997,934 the disclosures of which are incorporated herein by reference.

In an exemplary embodiment of the invention, a pressure sensitive valve according to one of the exemplary embodiments described above (e.g. valve 300) is installed on an atherectomy catheter behind the working head. As the working head traverses the stenosis, the valve is brought into proximity with the stenosis. Medication can be injected into vessel wall 310 and/or stenosis 320 as described above. Optionally, a catheter with imaging capabilities is used to align holes 290 with a desired target.

Exemplary Force Diagrams

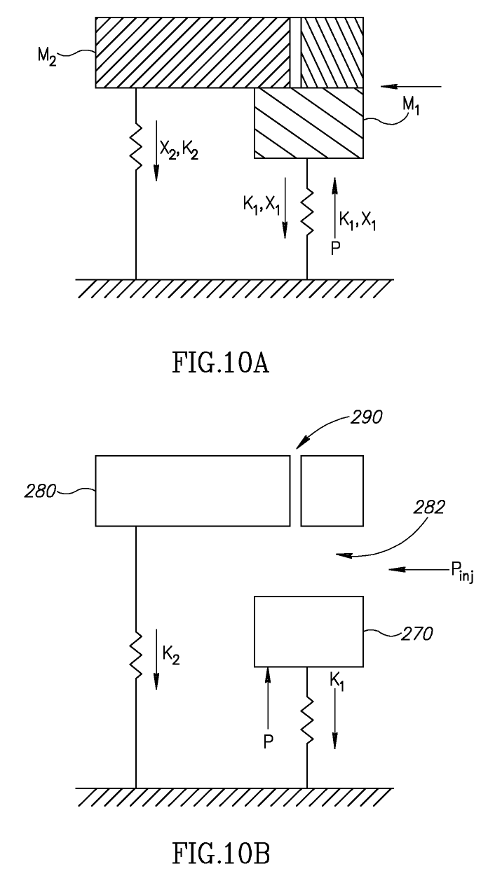

FIGS. 10A and 10B are diagrams illustrating an exemplary interplay of forces in an exemplary valve according to the invention. X is used here to indicate a constant.

FIG. 10A illustrates a theoretical interplay of forces between two masses M1 and M2, each supported by a separate spring and M2 partially resting on M1. As illustrated, an expansive pressure P provided by the spring of M1 can be expressed as Kn*Xn. The mass of M1 provides an opposing force with a magnitude K1*X1. In the depicted configuration M1 and M2 are in a steady state so that a downward force exerted by M2 on its spring (K2*X2) is equal to P−[K1*X1].

FIG. 10B shows an analogous situation with outer balloon 280 replacing M2 and inner balloon 270 replacing M1 the “springs” in this diagram represent the contractive force supplied by the coefficient of elasticity K of each balloon. Inner balloon 270 is inflated with a pressure P which is partially overcome by K1 of the inner balloon. When inner balloon 270 is inflated so that it conforms to outer balloon 280, K2 of the outer balloon is equal to P−[K1]. According to the depicted embodiment, Pᵢₙⱼ will be any pressure sufficient to overcome K2 and cause balloon 280 to move away from balloon 270 so that lumen 282 expands to permit a flow of fluid outwards from hole(s) 290.

A variety of numerical indicators have been utilized to describe dimensions of various components of the apparatus and/or operational pressures. These numerical indicators are exemplary only and could vary even further based upon a variety of engineering principles, materials, intended use and designs incorporated into the invention.

In addition individual features described herein can be used together, separately or in various sub-combinations. Alternatively or additionally, features described in the context of an apparatus may be applied to a method, and features described in the context of a method may be applied to an apparatus.

In an exemplary embodiment of the invention, an apparatus according to the invention is supplied as a kit including instructions for use and/or a medication. Optionally, the medication is provided as a pre-measured dose. Optionally, the pre-measure dose is pre-loaded into a catheter lumen and/or pulse gun. In an exemplary embodiment of the invention, use of an apparatus as described above reduces waste of medication. The examples presented are not intended to limit the scope of the invention, which is defined by the following claims.

The terms “include”, “comprise” and “have” and their conjugates as used herein mean “including but not necessarily limited to”.

1. A pressure sensitive valve, the valve comprising:
(a) an outer balloon adapted for intravascular insertion comprising at least one hole adapted for ejection of a fluid;
(b) an inner structure adapted to substantially fill the outer balloon;
(c) at least one selectively blockable flow path between the outer balloon and the inner structure, at least some of the at least one flow path in fluid communication with at least one of the at least one hole;
(d) an inlet port to the at least one flow path; and
(e) a pressure source operable to provide a fluid at least at a selected injection pressure to the inlet port;
wherein a flow of the fluid along the at least one selectively blockable flow path to the at least one hole is prevented when the pressure source provides any pressure below the selected injection pressure; and
wherein a flow of the fluid along the at least one selectively blockable flow path to the at least one hole occurs when the pressure source provides pressure at or above the selected injection pressure.

2. A valve according to claim 1, wherein the outer balloon is elastic.

3. A valve according to claim 2, wherein a coefficient of elasticity “K” of the outer balloon is at least 500 N/mm.

4. A valve according to claim 1, wherein the inner structure comprises a balloon.

5. A valve according to claim 4, wherein the balloon is elastic.

6. A valve according to claim 5, wherein a coefficient of elasticity “K” of the outer balloon is at least 100% greater than a coefficient of elasticity of the inner balloon.

7. A valve according to claim 1, wherein the outer balloon conforms to the inner structure at any pressure below the selected injection pressure.

8. A valve according to claim 7, wherein the outer balloon expands when pressure at the inlet port reaches or exceeds the selected injection pressure.

9. An atherectomy catheter comprising a valve according to claim 1.

10. A method of delivering fluid to tissue surrounding an intrabody lumen at a high velocity, the method comprising:
(a) inserting a pressure sensitive valve into an intrabody lumen, the valve configured to prevent a flow of fluid from one or more holes at any pressure below a selected injection pressure and to permit the flow at the selected injection pressure or greater; and
(b) delivering a fluid pulse to the valve at the selected injection pressure or greater.

11. A method according to claim 10, wherein the valve is adjacent to one or more holes.

12. A method according to claim 10, wherein the valve comprises one or more holes.

13. A method according to claim 10, wherein the fluid pulse comprises a liquid medication.

14. A method according to claim 10, wherein exit of a volume not exceeding 0.25 ml reduces pressure in the valve below the selected injection pressure.

15. A method according to claim 10, wherein the selected injection pressure is at least 10 atmospheres.

16. A method according to claim 10, wherein delivering fluid to the valve at the selected injection pressure or greater is repeated and the flow is prevented between repetitions.

17. A method according to claim 16, comprising adjusting an ejection direction between repetitions.

18. A method according to claim 16, comprising adjusting a position of the valve between the repetitions.

19. A method according to claim 10, performed in conjunction with a stenosis therapy procedure.

20. A method according to claim 19, wherein the stenosis therapy procedure comprises atherectomy.

21. A method according to claim 19, wherein the stenosis therapy procedure comprises PTCA.

22. A method of delivering fluid to tissue surrounding an intrabody lumen at a high velocity, the method comprising:
(a) inserting a pressure sensitive valve comprising an outer balloon with one or more holes and an inner balloon into an intrabody lumen;
(b) inflating the inner balloon so that it conforms to the outer balloon; and
(c) causing fluid to flow into a lumen of the outer balloon at a sufficient pressure to cause at least a portion of the fluid to exit the balloon through the holes at a velocity sufficient to penetrate surrounding tissue while the inner balloon remains inflated.

23. A method according to claim 22, wherein (a) occurs first, (b) occurs second and (c) occurs third.

24. A method according to claim 22, comprising inflating the inner balloon to open a stenosis.

25. A method according to claim 24, reducing pressure in the inner balloon after opening the stenosis.

26. A method of delivering fluid to tissue surrounding an intrabody lumen at a high velocity, the method comprising:
(a) stopping molecules of liquid propelled by an increasing pressure approaching a selected injected pressure, the increasing pressure supplied from a pressure source outside the body, using a pressure sensitive valve installed in a body lumen; and
(b) opening an acceleration path for the molecules when the increasing pressure reaches or exceeds the selected injection pressure

27. A method according to claim 26, wherein the valve stops the molecules within the valve.

28. A method according to claim 26, wherein the valve stops the molecules prior to entry into the valve.

29. A method according to claim 26, wherein opening the acceleration path comprises stretching an elastic membrane.

30. A method according to claim 29, wherein stretching an elastic membrane comprises expanding an elastic balloon.

31. A method according to claim 26, wherein opening the acceleration path comprises deforming a plastically deformable element.

32. A method according to claim 26, wherein opening the acceleration path comprises deforming an elastically deformable element.

33. A method according to claim 26, wherein opening the acceleration path comprises opening one or more elongate channels by operating one or more microvalves which open at the selected injection pressure.

34. A pressure sensitive valve, the valve comprising:
(a) a biocompatible unit, the unit adapted for insertion in an intrabody lumen;
(b) at least one acceleration path for a fluid, each of the at least one acceleration path terminating in at least one hole facing outwards from the biocompatible unit;
(c) an inlet port to the at least one flow path,
(d) a pressure source operable to provide fluid at a selected injection pressure to the inlet port; and
(e) at least one flow restriction element adapted to:
(i) block a flow of the fluid along the at least one flow path at any pressure below the selected injection pressure; and
(ii) permit a flow of the fluid along the at least one flow path at the selected injection pressure or greater.

35. A liquid drug delivery device for treating intra-body lumen tissues, the device comprising:
an inflatable inner structure containing a fluid at a substantially constant inner threshold pressure;
an outer structure having at least one hole, adapted for ejecting the drug;
a volume between said structures containing a liquid drug at a first pressure, which is substantially equivalent to said threshold pressure; and
a pressure pulse source having direct communication with said volume, adapted to substantially increase the pressure in said volume, when activated, for a period not exceeding 100 milliseconds;
wherein said inner structure seals said at least one hole when said pressure pulse source is not activated.
